(19) United States
(12) Patent Application Publication
(10) Pub. No.: US 2024/0390352 A1
(43) Pub. Date: Nov. 28, 2024

(54) PHARMACEUTICAL COMPOSITION FOR PREVENTING OR TREATING CANCER COMPRISING CARNITINE ACYLCARNITINE CARRIER INHIBITOR AND ANTITUMOR AGENT

(71) Applicant: NEW CANCER CURE - BIO CO.,LTD., Goyang-si, Gyeonggi-do (KR)

(72) Inventor: Soo Youl KIM, Goyang-si (KR)

(73) Assignee: NEW CANCER CURE - BIO CO.,LTD., Goyang-si, Gyeonggi-do (KR)

(21) Appl. No.: 18/694,721

(22) PCT Filed: Sep. 23, 2022
(86) PCT No.: PCT/KR2022/014279

§ 371 Date: Mar. 22, 2024

(30) Foreign Application Priority Data
Sep. 23, 2021 (KR) 10-2021-0125561
Sep. 23, 2022 (KR) 10-2022-0120696

(51) Int. Cl. A61K 31/4439; A61K 31/337; A61K 31/4745; A61K 31/513; A61K 31/7068; A61K 33/243; A61P 35/00

(52) U.S. Cl. CPC A61K 31/4439; A61K 31/337; A61K 31/4745; A61K 31/513; A61K 31/7068; A61K 33/243; A61P 35/00

(57) ABSTRACT

The present invention relates to a pharmaceutical composition for the prevention or treatment of cancer, including carnitine acylcarnitine carrier (CAC) inhibitor and an anticancer agent.

The compositions comprising the carnitine acylcarnitine carrier inhibitor and the anticancer agent of the present invention not only significantly reduced the growth of various cancer cells compared to the use of the carnitine acylcarnitine carrier inhibitor or the anticancer agent alone, but also confirmed the anti-cancer synergistic effect of co-administration of the carnitine acylcarnitine carrier inhibitor and the anticancer agent in a xenograft tumor animal model. Thus, the composition of the present invention is useful as effective combination anticancer agent.

### TECHNICAL FIELD

The present invention relates to a pharmaceutical composition for the prevention or treatment of cancer, including carnitine acylcarnitine carrier (CAC) inhibitor and an anticancer agent.

### BACKGROUND ART

While normal cells are capable of regular, resilient proliferation and suppression as needed, cancer cells have unrestricted proliferation, resulting in a cell mass composed of undifferentiated cells, also known as a tumor. These cancer cells infiltrate the surrounding tissues and metastasize to other organs of the body, causing severe suffering and eventually death. Despite medical advances, the number of cancer patients in Korea has continued to increase, increasing by about 44% in the last decade, and the market for anti-cancer drugs has also increased internationally, with a reported annual value of about $100 billion.

The first generation of anti-cancer drugs are chemical anti-cancer drugs, the second generation are targeted anti-cancer drugs, and immune anti-cancer drugs are being developed as the third generation of anti-cancer drugs to overcome their side effects. However, the biggest problem in current cancer treatment is cancer recurrence because it is difficult to target specific cancers due to various mutations in cancer, and it is not uncommon for recurrent cancers to develop resistance to the anticancer drugs used in the treatment process. As a result, even after the primary cancer is treated, patients often die from metastatic and recurrent cancers. Therefore, in order to improve the effectiveness of anticancer drugs, strategies have been proposed to combine anticancer drugs in combination.

Metabolic anti-cancer drugs are commonly called ‘therapeutics that starve cancer cells’ because they inhibit the metabolic process that cancer cells use for energy. They can inhibit cancer by targeting cancer cell-specific mitochondrial metabolism, metabolic vulnerability of cancer cells, and energy utilization processes of cancer cells in nutritional deficiency. In addition, at a time when anticancer treatment is challenging due to tumor mutations and acquisition of resistance, anticancer drugs that target cancer cell energy metabolism are showing new promise in solving the problems of anticancer drug resistance and cancer recurrence, and it is suggested that anticancer drugs will show the best efficiency in combination therapy with other anticancer drugs due to their advantages in overcoming anticancer drug resistance (Alba Luengo et al., Cell Chem Biol: 1161-1180, 2017).

Meanwhile, omeprazole, a carnitine acylcarnitine carrier (CAC) inhibitor, is a proton-pump inhibitor that has been shown to be effective in the treatment of gastroesophageal reflux disease, peptic ulcers, erosive esophagitis, or eosinophilic esophagitis. A recent study showed that proton-pump inhibitors, including omeprazole, inhibit the activity of Fatty Acid Synthase (FASN), which helps cancer cells produce fatty acids that are key to their survival, and induce cancer cell death with minimal effect on non-cancerous cells (Walsh et al., Journal of Experimental & Clinical Cancer Research, 34:93, 2015).

### DISCLOSURE

### Technical Problem

Efforts are being made to reduce the side effects of chemotherapy and increase the anticancer effect by co-administering carnitine acylcarnitine carriers, including omeprazole, as adjuvants.

Accordingly, the present inventors have made a conscientious effort to provide a combination anticancer agent that can significantly inhibit cancer cells, and have confirmed that treatment with a carnitine acylcarnitine carrier inhibitor and an anticancer agent in combination significantly increases the inhibitory effect of cancer cells compared to treatment with each of them alone, and have completed the present invention.

Accordingly, it is an object of the present invention to provide a pharmaceutical composition for the prevention or treatment of cancer comprising a carnitine acylcarnitine carrier inhibitor and an anticancer agent as active ingredients.

Another object of the present invention is to provide an anticancer adjuvant comprising a carnitine acylcarnitine carrier inhibitor and an anticancer agent as active ingredients.

### Technical Solution

To fulfill the purposes described above, the present invention provides a pharmaceutical composition for the prevention or treatment of cancer comprising a Carnitine Acylcarnitine Carrier (CAC) inhibitor and an anticancer agent as active ingredients.

The present invention also provides an anti-cancer adjuvant comprising a carnitine acylcarnitine carrier inhibitor and an anti-cancer agent as active ingredients.

According to a preferred embodiment of the present invention, the carnitine acylcarnitine carrier inhibitor may be one or more selected from the group consisting of omeprazole (KN510), lansoprazole (KN511), pantoprazole (KN512) and pharmaceutically acceptable salts thereof.

According to another preferred embodiment of the present invention, the anticancer agent may be one or more selected from the group consisting of Irinotecan, fluorouracil (5-FU), Paclitaxel, Gemcitabine, Cisplatin, and pharmaceutically acceptable salts thereof.

According to another preferred embodiment of the present invention, the carnitine acylcarnitine carrier inhibitor and the anticancer agent may be included in a concentration ratio of 100:0.1 to 1:1, preferably in a concentration ratio of 100:0.1 to 100:10, more preferably in a concentration ratio of 100:0.5 to 100:5.

According to another preferred embodiment of the present invention, the carnitine acylcarnitine carrier inhibitor and the anticancer agent may be administered sequentially or simultaneously.

According to another preferred embodiment of the present invention, the cancer may be any one or more cancers selected from the group consisting of colorectal cancer, lung cancer, stomach cancer, breast cancer, melanoma, leukemia, ovarian cancer, renal cancer, pancreatic cancer, glioblastoma, and liver cancer.

According to another preferred embodiment of the present invention, wherein when the anticancer agent is irinotecan or a pharmaceutically acceptable salt thereof, the cancer is colon cancer, renal cancer, liver cancer, glioblastoma, pancreatic cancer, breast cancer, or leukemia;

when the anticancer agent is paclitaxel or a pharmaceutically acceptable salt thereof, the cancer is colon cancer, glioblastoma, melanoma, pancreatic cancer, stomach cancer, lung cancer, or leukemia;

when the anticancer agent is 5-FU (fluorouracil) or a pharmaceutically acceptable salt thereof, the cancer is colon cancer, renal cancer, liver cancer, melanoma, lung cancer, or leukemia; and/or when the anticancer agent is cisplatin or a pharmaceutically acceptable salt thereof, the cancer may be ovarian cancer or leukemia.

### Advantageous Effects

The composition comprising the carnitine acylcarnitine carrier inhibitor and the anticancer agent of the present invention not only significantly reduced the growth of various cancer cells compared to the use of the carnitine acylcarnitine carrier inhibitor or the anticancer agent alone, but also confirmed the anti-cancer synergistic effect of co-administration of the carnitine acylcarnitine carrier inhibitor and the anticancer agent in a xenograft tumor animal model. Thus, the composition of the present invention is useful as an effective combination anticancer agent.

### DESCRIPTION OF DRAWINGS

FIG. 1 shows data on cancer cell growth rates when colon cancer cell lines HCT-116 cells and COLO-205 cells were treated with omeprazole (KN510) and/or various anticancer drugs (irinotecan, 5-FU, paclitaxel, gemcitabine, cisplatin) alone or in combination. FIG. 1a shows the data for drugs that show synergistic anti-cancer effect by co-administration, and FIG. 1b shows the data for drugs that do not show synergistic anti-cancer effect by co-administration.

FIG. 2 shows data on cancer cell growth rates of CAKI-1 and ACHN cells, renal cancer cell lines, when treated with omeprazole (KN510) and/or various anticancer drugs (irinotecan, 5-FU, paclitaxel, gemcitabine, cisplatin) alone or in combination. FIG. 2a shows the data for drugs that show synergistic anti-cancer effect by co-administration, and FIG. 2b shows the data for drugs that do not show synergistic anti-cancer effect by co-administration.

FIG. 3 shows data on cancer cell growth rates when liver cancer cell lines, SK-HEP-1 cells and Huh-7 cells, were treated with omeprazole (KN510) and/or various anticancer drugs (irinotecan, 5-FU, paclitaxel, gemcitabine, cisplatin) alone or in combination. FIG. 3a shows the data for drugs that showed synergistic anti-cancer effects in combination, and FIG. 3b shows the data for drugs that did not show synergistic anti-cancer effects in combination.

FIG. 4 shows data on cancer cell growth rates when glioblastoma (GBM) cell lines, U-87 MG cells and T87G cells, were treated with omeprazole (KN510) and/or various anticancer drugs (irinotecan, 5-FU, paclitaxel, gemcitabine, cisplatin) alone or in combination. FIG. 4a shows the data for drugs that show synergistic anti-cancer effect by co-administration, and FIG. 4b shows the data for drugs that do not show synergistic anti-cancer effect by co-administration.

FIG. 5 shows data on cancer cell growth rates when melanoma cell lines UACC62 cells and UACC257 cells were treated with omeprazole (KN510) and/or various anticancer drugs (irinotecan, 5-FU, paclitaxel, gemcitabine, cisplatin) alone or in combination. FIG. 5a shows the data for drugs that show synergistic anti-cancer effect by co-administration, and FIG. 5b shows the data for drugs that do not show synergistic anti-cancer effect by co-administration.

FIG. 6 shows data on cancer cell growth rates when pancreatic ductal adenocarcinoma (PDAC) cell lines, MIA PaCa-2 cells and PANC-1 cells, were treated with omeprazole (KN510) and/or various anticancer drugs (irinotecan, 5-FU, paclitaxel, gemcitabine, cisplatin) alone or in combination. FIG. 6a shows the data for drugs that show synergistic anti-cancer effect by co-administration, and FIG. 6b shows the data for drugs that do not show synergistic anti-cancer effect by co-administration.

FIG. 7 shows data on cancer cell growth rates when MKN-28 cells and AGS cells, which are stomach cancer cells, were treated with omeprazole (KN510) and/or various anticancer drugs (irinotecan, 5-FU, paclitaxel, gemcitabine, cisplatin) alone or in combination. FIG. 7a shows the data for drugs that show synergistic anti-cancer effect by co-administration, and FIG. 7b shows the data for drugs that do not show synergistic anti-cancer effect by co-administration.

FIG. 8 shows data on cancer cell growth rates when ovarian cancer cells, OVCAR-8 cells and SK-OV-3 cells, were treated with omeprazole (KN510) and/or various anticancer drugs (irinotecan, 5-FU, paclitaxel, gemcitabine, cisplatin) alone or in combination. FIG. 8a shows the data for drugs that show synergistic anti-cancer effect by co-administration, and FIG. 8b shows the data for drugs that do not show synergistic anti-cancer effect by co-administration.

FIG. 9 shows data on cancer cell growth rates when lung cancer cells, A549 cells and H23 cells, were treated with omeprazole (KN510) and/or various anticancer drugs (irinotecan, 5-FU, paclitaxel, gemcitabine, cisplatin) alone or in combination. FIG. 9a shows the data for drugs that show synergistic anti-cancer effect by co-administration, and FIG. 9b shows the data for drugs that do not show synergistic anti-cancer effect by co-administration.

FIG. 10 shows data on cancer cell growth rates when breast cancer cells, MDA-MB-231 cells and MCF-7 cells, were treated with omeprazole (KN510) and/or various anticancer drugs (irinotecan, 5-FU, paclitaxel, gemcitabine, cisplatin) alone or in combination. FIG. 10a shows the data for drugs that show synergistic anti-cancer effect by co-administration, and FIG. 10b shows the data for drugs that do not show synergistic anti-cancer effect by co-administration.

FIG. 11 shows data on cancer cell growth rates in prostate cancer cells, PC-3 and DU-145 cells, when treated with omeprazole (KN510) and/or various anticancer drugs (irinotecan, 5-FU, paclitaxel, gemcitabine, cisplatin) alone or in combination. In prostate cancer, all combinations did not show synergistic effects.

FIG. 12 shows data on cancer cell growth rates when SR cells and K562 cells, which are Leukemia cells, were treated with omeprazole (KN510) and/or various anticancer drugs (irinotecan, 5-FU, paclitaxel, gemcitabine, cisplatin) alone or in combination. FIG. 12a shows the data for drugs that show synergistic anti-cancer effect by co-administration, and FIG. 12b shows the data for drugs that do not show synergistic anti-cancer effect by co-administration.

FIG. 13 shows data observing changes in tumor tissue size (a) when omeprazole (KN510, 50 mg/kg) and irinotecan (20 mg/kg) were treated alone or in combination in a pancreatic cancer xenograft tumor model, and (b) when omeprazole (KN510, 100 mg/kg) and irinotecan (20 mg/kg) were treated alone or in combination in a pancreatic cancer xenograft tumor model.

### MODE OF THE INVENTION

The present invention will now be described in detail.

In one aspect, the present invention relates to a pharmaceutical composition for the prevention or treatment of cancer comprising a Carnitine Acylcarnitine Carrier (CAC) inhibitor and an anticancer agent as active ingredients.

In another aspect, the present invention relates to an anti-cancer adjuvant comprising a carnitine acylcarnitine carrier inhibitor and an anti-cancer agent as active ingredients.

In the present invention, the carnitine acylcarnitine carrier inhibitor may be one or more selected from the group consisting of omeprazole (KN510), lansoprazole (KN511), pantoprazole (KN512) and pharmaceutically acceptable salts thereof, preferably omeprazole or pharmaceutically acceptable salts thereof.

In the present invention, the anticancer agent may be a metabolic inhibitor or a metabolic anticancer agent, preferably one or more selected from the group consisting of Irinotecan, fluorouracil (5-FU), Paclitaxel, Gemcitabine, Cisplatin and pharmaceutically acceptable salts thereof.

In the present invention, the carnitine acylcarnitine carrier inhibitor and the anticancer agent may be administered sequentially or simultaneously.

In the present invention, the carnitine acylcarnitine carrier inhibitor and the anticancer agent may be included in a concentration ratio of 100:0.1 to 1:1, preferably in a concentration ratio of 100:0.1 to 100:10, more preferably in a concentration ratio of 100:0.5 to 100:5.

In the present invention, the cancer may be any one or more cancers selected from the group consisting of colon cancer, lung cancer, stomach cancer, breast cancer, melanoma, leukemia, ovarian cancer, renal cancer, pancreatic cancer, glioblastoma, and liver cancer.

Where the anti-cancer agent is irinotecan or a pharmaceutically acceptable salt thereof, the cancer may be colon cancer, renal cancer, liver cancer, glioblastoma, pancreatic cancer, breast cancer, or leukemia,
where the anti-cancer agent is paclitaxel or a pharmaceutically acceptable salt thereof, and the cancer may be colorectal cancer, glioblastoma, melanoma, pancreatic cancer, stomach cancer, lung cancer, or leukemia,
where the anti-cancer agent is 5-FU (fluorouracil) or a pharmaceutically acceptable salt thereof, and the cancer may be colon cancer, renal cancer, liver cancer, melanoma, lung cancer, or leukemia,
wherein the anti-cancer agent is cisplatin or a pharmaceutically acceptable salt thereof, the cancer may be ovarian cancer or leukemia.

In a specific embodiment of the present invention, for colon cancer, renal cancer, liver cancer, glioblastoma, melanoma, pancreatic cancer, stomach cancer, ovarian cancer, lung cancer, breast cancer, prostate cancer and leukemia-derived cell lines, anticancer drugs that showed synergistic anticancer effects were selected when omeprazole (KN510), and irinotecan, paclitaxel, 5-FU, gemcitabine or cisplatin as an anticancer drug were administered in combination.

TABLE 1
Identify synergistic effects of omeprazole
and an anticancer drug in combination

| Cancer | | Drug |
|---|---|---|
| Colon | Positive effect | Irinotecan, Paclitaxel, 5-FU |
| Cancer | Negative effect | Gemcitabine, Cisplatin |
| Renal cell | Positive effect | Irinotecan, 5-FU |
| Carcinoma | Negative effect | Gemcitabine, Paclitaxel, Cisplatin |
| Liver | Positive effect | Irinotecan, 5-FU |
| Cancer | Negative effect | Gemcitabine, Paclitaxel, Cisplatin |
| Glioblastoma | Positive effect | Irinotecan, Paclitaxel |
| (GBM) | Negative effect | 5-FU, gemcitabine, cisplatin |
| Melanoma | Positive effect | Irinotecan, 5-FU, Paclitaxel |
| | Negative effect | Gemcitabine, Cisplatin |
| Pancreatic | Positive effect | Irinotecan, Paclitaxel |
| cancer | Negative effect | 5-FU, gemcitabine, cisplatin |
| (PDAC) | | |
| Stomach | Positive effect | Paclitaxel |
| Cancer | Negative effect | Irinotecan, 5-FU, Gemcitabine, Cisplatin |
| Ovarian | Positive effect | Cisplatin |
| Cancer | Negative effect | Irinotecan, 5-FU, Paclitaxel, Gemcitabine |
| Lung | Positive effect | 5-FU, Paclitaxel |
| Cancer | Negative effect | Irinotecan, gemcitabine, cisplatin |
| Breast | Positive effect | Irinotecan |
| Cancer | Negative effect | 5-FU, gemcitabine, paclitaxel, cisplatin |
| Prostate | Positive effect | —. |
| Cancer | Negative effect | Irinotecan, 5-FU, gemcitabine, paclitaxel, |
| | | cisplatin |
| Leukemia | Positive effect | Irinotecan, 5-FU, Paclitaxel, Cisplatin |
| | Negative effect | Gemcitabine |

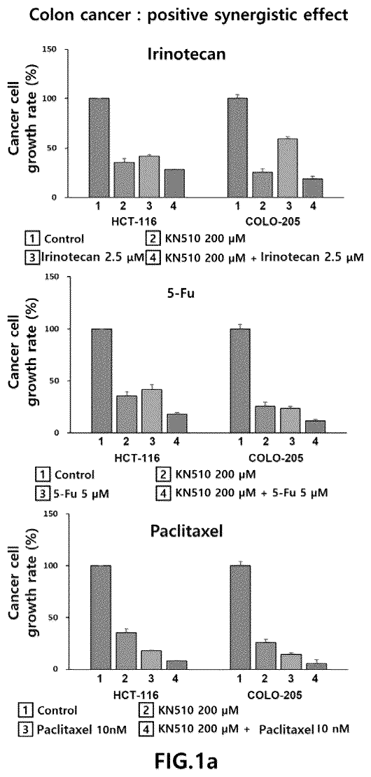

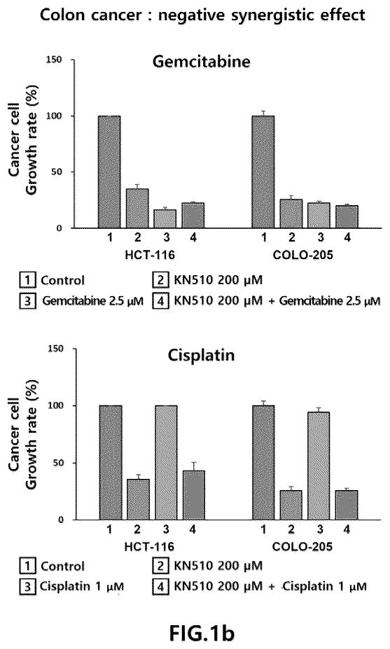

As a result, as shown in Table 1 and FIGS. 1 to 9 above, the combination of omeprazole (KN510)+irinotecan significantly increased cancer cell killing in colon cancer, renal cancer, liver cancer, glioblastoma, pancreatic cancer, breast cancer, and leukemia cell lines compared to each treatment alone;
the combination of omeprazole (KN510)+paclitaxel significantly increased cancer cell killing in colorectal cancer, glioblastoma, melanoma, pancreatic cancer, stomach cancer, lung cancer, and leukemia cell lines compared to each treatment alone;
the combination of omeprazole (KN510)+5-FU significantly increased cancer cell killing in colon cancer, renal cancer, liver cancer, melanoma, lung cancer, and leukemia cell lines compared to each treatment alone; and
the combination of omeprazole (KN510)+cisplatin significantly increased cancer cell killing in ovarian cancer and leukemia cell lines compared to each treatment alone.

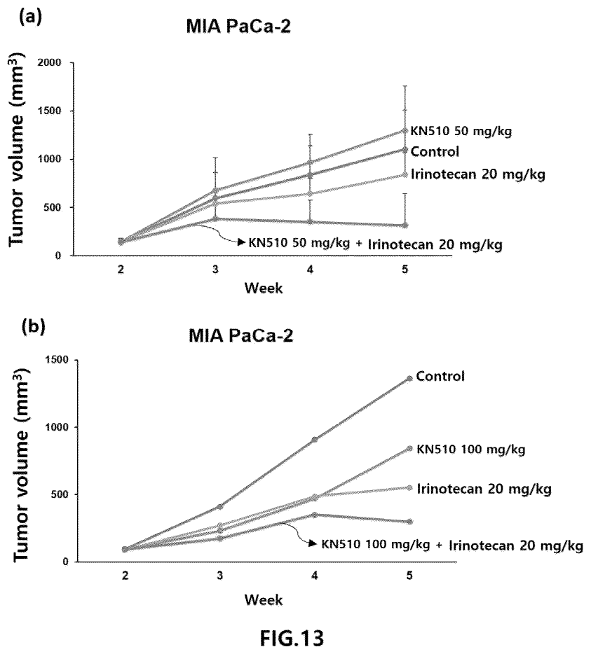

In another specific embodiment of the present invention, to determine the anti-cancer synergistic effect of omeprazole (KN510) and an anticancer drug in vivo, a pancreatic cancer xenograft tumor model was established, and then omeprazole (KN510, 50 mg/kg or 100 mg/kg) and irinotecan (20 mg/kg) were administered alone or in combination. As shown in FIG. 13, a significant reduction in tumor size was observed when omeprazole (KN510) and irinotecan were treated in combination compared to each treatment alone.

The composition of the present invention can be in a number of different formulations, either oral or parenteral. The composition may be formulated using one or more buffers (e.g., saline or PBS), antioxidants, bacteriostatic agents, chelating agents (e.g., EDTA or glutathione), fillers, bulking agents, binders, auxiliaries (e.g., aluminum hydroxide), suspending agents, thickening agents wetting agents, disintegrating agents or surfactants, diluents, or excipients.

Solid dosage forms for oral administration include tablets, pills, powders, granules, capsules, and the like, wherein one or more compounds are combined with at least one excipient, such as starch (including corn starch, wheat starch, rice starch, potato starch, and the like), calcium carbonate, sucrose, lactose, dextrose, sorbitol, mannitol, mannitol, xylitol, erythritol maltitol, cellulose, methyl cellulose, sodium carboxymethylcellulose and hydroxypropylmethyl-cellulose, or gelatin. For example, tablets or saccharide tablets can be obtained by mixing the active ingredient with solid excipients, grinding it, adding suitable excipients and processing it into a granular mixture.

In addition to simple excipients, lubricants such as magnesium stearate, talc, etc. are also used. Liquid preparations for oral administration include suspensions, solutions, emulsions, or syrups, which may contain a variety of excipients, such as wetting agents, sweeteners, flavors, or preservatives, in addition to the commonly used simple diluents of water and liquid paraffin. In addition, in some cases, cross-linked polyvinylpyrrolidone, agar, alginate or sodium alginate may be added as a disintegrating agent, and may further include anti-flocculants, lubricants, wetting agents, flavors, emulsifiers, and preservatives.

Formulations for parenteral administration include sterile aqueous solutions, non-aqueous solutions, suspensions, emulsions, lyophilizates, or suppositories. Non-aqueous solvents and suspensions may include propylene glycol, polyethylene glycol, vegetable oils such as olive oil, and injectable esters such as ethylolate. As a base for suppositories, witepsol, macrogol, tween 61, cacao paper, laurin paper, glycerol, gelatin, etc. can be used.

The composition of the invention may be administered orally or parenterally and, when administered parenterally, may be formulated according to methods known in the art in the form of an injectable for topical use; intraperitoneal, rectal, intravenous, intramuscular, subcutaneous, intrauterine, intrathecal, or intracerebral injection.

In the case of the injectable, it must be sterile and protected from contamination by microorganisms such as bacteria and fungi. Examples of suitable carriers for injectables include, but are not limited to, solvents or dispersion media comprising water, ethanol, polyols (e.g., glycerol, propylene glycol, and liquid polyethylene glycol), mixtures thereof, and/or vegetable oils. More preferably, suitable carriers include Hanks' solution, Ringer's solution, phosphate buffered saline (PBS) containing triethanolamine, or isotonic solutions such as sterile water for injection, 10% ethanol, 40% propylene glycol, and 5% dextrose. To protect the injectable from microbial contamination, the injectable may additionally contain various antibacterial and antifungal agents such as parabens, chlorobutanol, phenols, sorbic acid, thimerosal, and the like. In addition, the injectable may further comprise an isotonicizing agent such as sugar or sodium chloride in most cases.

The composition of the invention can be administered in a pharmaceutically effective amount. A pharmaceutically effective amount means an amount sufficient to treat a condition with a reasonable benefit/risk ratio applicable to medical treatment, and the effective dose level may be determined by factors including the type and severity of the patient's condition, the activity of the drug, sensitivity to the drug, time of administration, route of administration and elimination rate, duration of treatment, concomitant medications, and other factors well known in the medical field. The composition of the present invention may be administered as individual therapeutic agents or in combination with other therapeutic agents, may be administered sequentially or simultaneously with conventional therapeutic agents, and may be administered in single or multiple doses, i.e., the total effective amount of the composition of the present invention may be administered to the patient in a single dose, or may be administered in a fractionated treatment protocol in which multiple doses are administered over a longer period of time. Taking all of the above factors into consideration, it is important to administer an amount that provides maximum benefit in a minimal amount without side effects, which can be readily determined by those skilled in the art.

The preferred dosage of the composition depends on the condition of the patient, body weight, severity of the disease, drug form, route of administration and duration, but may be suitably selected by those skilled in the art, for example from 0.0001 to 2,000 mg/kg per day, more preferably from 0.001 to 2,000 mg/kg per day. The dose may be administered once daily or may be divided into several doses. However, the above dosages do not limit the scope of the invention.

The composition of the present invention can be used alone or in combination with surgery, radiation therapy, hormone therapy, chemotherapy, and methods using biological response modifiers.

The anticancer adjuvant of the present invention refers to any form of anti-cancer agent intended to increase the anti-cancer effect of an anti-cancer agent or to suppress or improve the side effects of an anti-cancer agent. The anti-cancer adjuvant of the present invention can be administered in combination with various types of anti-cancer drugs or anti-cancer adjuvants, and when administered in combination, the anti-cancer drug can be administered at a lower level than the conventional anti-cancer drug dosage, but still show an equivalent level of anti-cancer effect, thus enabling safer anti-cancer treatment.

The route of administration of the anticancer adjuvant may be by any conventional route as long as it can reach the target tissue. The anticancer adjuvant of the present invention may be administered by, but is not limited to, intraperitoneal, intravenous, intramuscular, subcutaneous, oral, intrapulmonary, or rectal administration, depending on the intended use. Furthermore, the anticancer adjuvant may be administered by any device capable of transporting the active substance to the target cell.

The anticancer adjuvant of the present invention can be preferably formulated as an anticancer adjuvant by including one or more pharmaceutically acceptable carriers in addition to the active ingredient for administration. Carriers, excipients, or diluents that may be included in the anticancer therapeutic adjuvants of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oils, including but not limited to.

The anticancer adjuvant of the present invention may be a formulation for oral or parenteral administration, and the description of the formulation is hereby superseded by the description of the pharmaceutical composition.

The present invention will now be described in more detail with reference to the following examples. These embodiments are for the purpose of illustrating the invention only, and it will be apparent to one of ordinary skill in the art that the scope of the invention is not to be construed as limited by these embodiments.

### Example 1: Confirmation of Anticancer Activity Against Colorectal Cancer by Coadministration

In the present invention, to determine the effect of co-treatment with Carnitine Acylcarnitine Carrier (CAC) inhibitor and an anticancer drug on cancer cell growth, the degree of cancer cell death was determined by Sulforhodamine B colorimetric assay (SRB assay), and the cancer cell growth rate was analyzed relative to the control (100%). All experiments were performed in triplicate, and data were expressed as mean (mean)+standard deviation (SD) values.

First, colon cancer cell lines HCT-116 cells and COLO-205 cells were prepared, and cells (100 μl) were inoculated into 96-well cell culture plates at plating densities ranging from 5,000 to 20,000 cells/well, depending on the doubling time of each cell line. After cell inoculation, the plates were incubated for 24 hours before the addition of the experimental drug. Omeprazole (KN510) and/or anticancer drugs were added to each well such that the concentrations were as shown in the following groups:
1) Control,
2) Omeprazole (KN510) 200 μM alone,
3) anticancer drug alone (irinotecan 2.5 μM, 5-FU 5 μM, paclitaxel 10 nM, gemcitabine 2.5 μM, or cisplatin 1 μM)
4) Omeprazole (KN510) 200 μM+anticancer drug (irinotecan 2.5 μM, 5-FU 5 μM, paclitaxel 10 nM, gemcitabine 2.5 M, or cisplatin 1 μM) as a combination treatment group

The cells were then incubated in a CO₂ incubator for 48 hours, after which the assay was terminated by the addition of cold trichloroacetic acid (TCA). Cells were fixed in situ by gently adding 50 μl of cold 50% (w/v) TCA (final concentration: 10% TCA) and incubated at 4° C. for 60 min. The supernatant was discarded, and the plate was washed five times with distilled water and air dried. A solution (100 μl) of 0.4% (w/v) Sulforhodamine B (SRB) in 1% acetic acid was added to each well, and the plate was left at room temperature for 10 min. After staining, unbound dye was removed by washing five times with 1% acetic acid, and the plate was air-dried. The bound dye was then solubilized with 10 mM trizma base, and the absorbance was recorded at 515 nm using an automated plate reader.

TABLE 2
Colon cancer cell line growth rates in response to drug treatment

| | | HCT-116 | | COLO-205 | |
|---|---|---|---|---|---|
| | | average | | average | |
| Drug | | (%) | SD | (%) | SD |
| Irinotecan | Control | 100.00 | 0.00 | 100.00 | 4.28 |
| | KN510 200 μM | 35.30 | 4.12 | 25.62 | 3.47 |
| | Irinotecan 2.5 μM | 41.85 | 1.59 | 59.34 | 1.93 |
| | KN510 200 μM + | 28.03 | 0.83 | 18.72 | 2.56 |
| | Irinotecan 2.5 μM | | | | |
| 5-FU | Control | 100.00 | 0.00 | 100.00 | 4.28 |
| | KN510 200 μM | 35.30 | 4.12 | 25.62 | 3.47 |
| | 5-FU 5 μM | 41.59 | 5.20 | 23.39 | 2.06 |
| | KN510 200 μM | 17.64 | 1.87 | 11.48 | 1.55 |
| | 5-FU 5 μM | | | | |
| Gemcitabine | Control | 100.00 | 0.00 | 100.00 | 4.28 |
| | KN510 200 μM | 35.30 | 4.12 | 25.62 | 3.47 |
| | Gemcitabine 2.5 μM | 16.32 | 2.37 | 22.24 | 1.93 |
| | KN510 200 μM + | 22.18 | 1.33 | 20.09 | 1.31 |
| | Gemcitabine 2.5 μM | | | | |
| Paclitaxel | Control | 100.00 | 0.00 | 100.00 | 4.28 |
| | KN510 200 μM | 35.30 | 4.12 | 25.62 | 3.47 |
| | Paclitaxel 10 nM | 17.70 | 0.54 | 14.57 | 1.34 |
| | KN510 200 μM + | 7.96 | 0.65 | 5.57 | 3.49 |
| | Paclitaxel 10 nM | | | | |
| Cisplatin | Control | 100.00 | 0.00 | 100.00 | 4.28 |
| | KN510 200 μM | 35.30 | 4.12 | 25.62 | 3.47 |
| | Cisplatin 1 μM | 100.00 | 0.00 | 94.22 | 4.25 |
| | KN510 200 μM + | 43.27 | 6.96 | 25.54 | 2.15 |
| | Cisplatin 1 μM | | | | |

As shown in FIG. 1 and Table 2, we found that when colon cancer cell lines were treated with irinotecan, paclitaxel, or 5-FU in combination with omeprazole (KN510), it was confirmed that colon cancer cell growth was significantly inhibited compared to the group treated alone.

### Example 2: Confirmation of Anti-Cancer Activity Against Renal Cancer by Co-Administration

Experiments were performed using the renal cell carcinoma cell lines CAKI-1 and ACHN cells in the same manner as in Example 1 above.

TABLE 3
Renal cancer cell line growth rate in response to drug treatment

| | | CAKI-1 | | ACHN | |
|---|---|---|---|---|---|
| | | average | | average | |
| Drug | | (%) | SD | (%) | SD |
| Irinotecan | Control | 100.00 | 6.37 | 100.00 | 9.42 |
| | KN510 200 μM | 41.69 | 2.10 | 17.28 | 0.54 |
| | Irinotecan 2.5 μM | 44.74 | 10.43 | 42.20 | 27.15 |
| | KN510 200 μM + | 28.59 | 2.14 | 8.71 | 0.28 |
| | Irinotecan 2.5 μM | | | | |
| 5-FU | Control | 100.00 | 6.37 | 100.00 | 9.42 |
| | KN510 200 μM | 41.69 | 2.10 | 17.28 | 0.54 |
| | 5-FU 5 μM | 47.91 | 4.73 | 48.97 | 4.47 |
| | KN510 200 μM | 24.21 | 4.93 | 7.34 | 0.78 |
| | 5-FU 5 μM | | | | |
| Gemcitabine | Control | 100.00 | 6.37 | 100.00 | 9.42 |
| | KN510 200 μM | 41.69 | 2.10 | 17.28 | 0.54 |
| | Gemcitabine 2.5 μM | 2.30 | 1.15 | −2.82 | 0.68 |
| | KN510 200 μM + | 24.15 | 1.05 | 3.76 | 0.35 |
| | Gemcitabine 2.5 μM | | | | |
| Paclitaxel | Control | 100.00 | 100.00 | 6.37 | 100.00 |
| | KN510 200 μM | 35.30 | 41.69 | 2.10 | 17.28 |
| | Paclitaxel 10 nM | 17.70 | 80.81 | 0.91 | 59.69 |
| | KN510 200 μM + | 7.96 | 42.29 | 4.19 | 14.88 |
| | Paclitaxel 10 nM | | | | |
| Cisplatin | Control | 100.00 | 6.37 | 100.00 | 9.42 |
| | KN510 200 μM | 41.69 | 2.10 | 17.28 | 0.54 |
| | Cisplatin 1 μM | 88.17 | 4.97 | 78.57 | 7.37 |
| | KN510 200 μM + | 56.04 | 5.94 | 18.46 | 1.12 |
| | Cisplatin 1 μM | | | | |

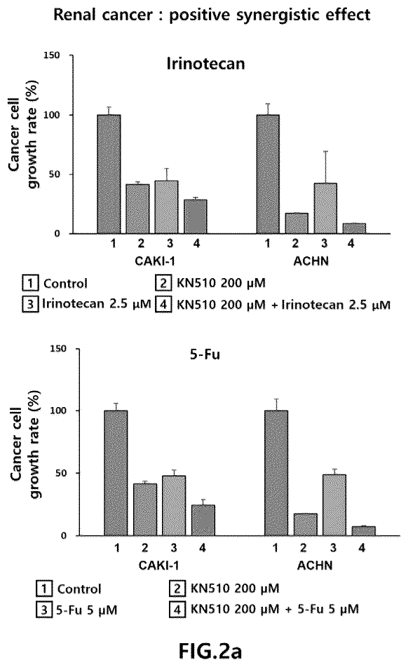

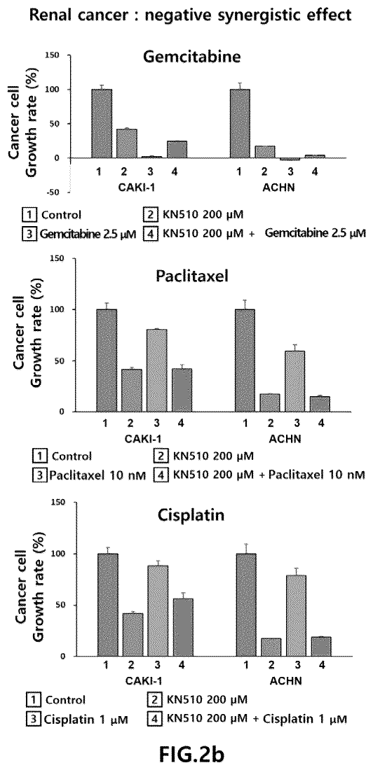

As shown in FIG. 2 and Table 3, when renal cancer cell lines were treated with irinotecan or 5-FU in combination with omeprazole (KN510), it was confirmed that renal cancer cell growth was significantly inhibited compared to the group treated alone.

### Example 3: Confirmation of Anti-Cancer Activity Against Liver Cancer by Co-Administration

Experiments were performed in the same manner as in Example 1 above using the liver cancer cell lines SK-HEP-1 cells and Huh-7 cells.

TABLE 4
Liver cancer cell line growth rate in response to drug treatment

| | | SK-HEP-1 | | Huh7 | |
|---|---|---|---|---|---|
| | | average | | average | |
| Drug | | (%) | SD | (%) | SD |
| Irinotecan | Control | 100.00 | 5.23 | 100.00 | 0.87 |
| | KN510 200 μM | 20.20 | 1.16 | 9.69 | 1.05 |
| | Irinotecan 2.5 μM | 47.04 | 2.55 | 42.82 | 3.26 |
| | KN510 200 μM + | 18.87 | 1.48 | 6.08 | 0.46 |
| | Irinotecan 2.5 μM | | | | |
| 5-FU | Control | 100.00 | 5.23 | 100.00 | 0.87 |
| | KN510 200 μM | 20.20 | 1.16 | 9.69 | 1.05 |
| | 5-FU 5 μM | 61.32 | 2.05 | 42.30 | 3.17 |
| | KN510 200 μM | 16.82 | 1.72 | 3.41 | 1.12 |
| | 5-FU 5 μM | | | | |
| Gemcitabine | Control | 100.00 | 5.23 | 100.00 | 0.87 |
| | KN510 200 μM | 20.20 | 1.16 | 9.69 | 1.05 |
| | Gemcitabine 2.5 μM | 21.64 | 1.06 | 22.98 | 0.90 |
| | KN510 200 μM + | 25.22 | 0.15 | 13.39 | 1.45 |
| | Gemcitabine 2.5 μM | | | | |
| Paclitaxel | Control | 100.00 | 5.23 | 100.00 | 0.87 |
| | KN510 200 μM | 20.20 | 1.16 | 9.69 | 1.05 |
| | Paclitaxel 10 nM | 32.17 | 0.45 | 32.69 | 0.30 |
| | KN510 200 μM + | 12.22 | 1.21 | 13.00 | 0.37 |
| | Paclitaxel 10 nM | | | | |
| Cisplatin | Control | 100.00 | 5.23 | 100.00 | 0.87 |
| | KN510 200 μM | 20.20 | 1.16 | 9.69 | 1.05 |
| | Cisplatin 1 μM | 91.86 | 3.39 | 53.37 | 3.46 |
| | KN510 200 μM + | 27.27 | 1.43 | 13.94 | 4.06 |
| | Cisplatin 1 μM | | | | |

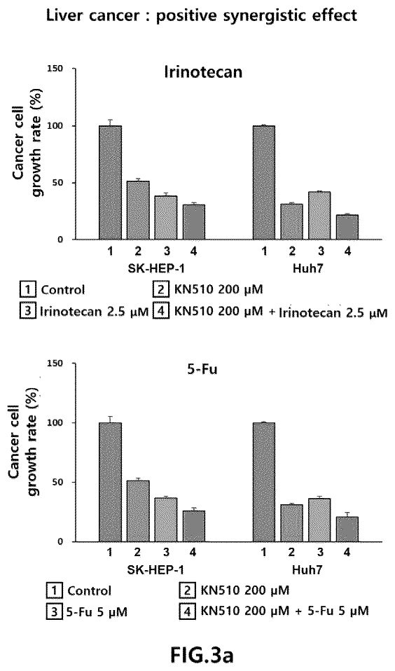

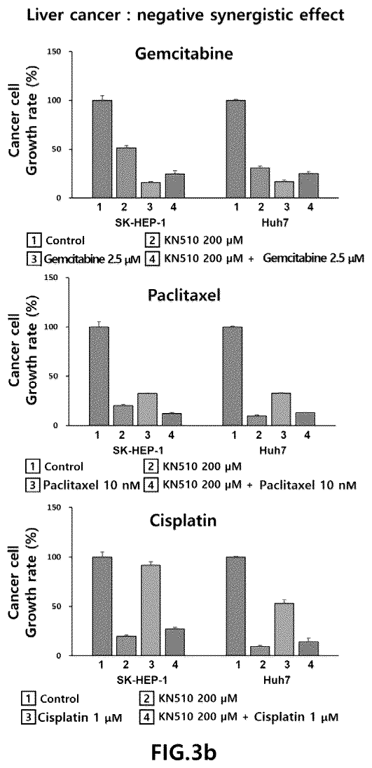

As shown in FIG. 3 and Table 4, when liver cancer cell lines were treated with irinotecan or 5-FU in combination with omeprazole (KN510), it was confirmed that cancer cell growth was significantly inhibited compared to the group treated alone.

### Example 4: Confirmation of Anti-Cancer Glioblastoma by Combination Dosing

Experiments were performed using glioblastoma (GBM) cell lines, U-87 MG cells and T87G cells, in the same manner as in Example 1 above.

TABLE 5
Glioblastoma cell line growth rate in response to drug treatment

| | | U-87 MG | | T87G | |
|---|---|---|---|---|---|
| | | average | | average | |
| Drug | | (%) | SD | (%) | SD |
| Irinotecan | Control | 100.00 | 0.00 | 100.00 | 6.14 |
| | KN510 200 μM | 57.73 | 8.05 | 24.31 | 3.03 |
| | Irinotecan 2.5 μM | 15.59 | 1.99 | 41.16 | 2.36 |
| | KN510 200 μM + | 8.39 | 0.37 | 10.86 | 0.20 |
| | Irinotecan 2.5 μM | | | | |
| 5-FU | Control | 100.00 | 0.00 | 100.00 | 1.62 |
| | KN510 200 μM | 57.73 | 8.05 | 32.53 | 2.08 |
| | 5-FU 5 μM | 9.93 | 0.65 | 76.71 | 4.27 |
| | KN510 200 μM | 9.06 | 0.67 | 26.25 | 5.21 |
| | 5-FU 5 μM | | | | |
| Gemcitabine | Control | 100.00 | 0.00 | 100.00 | 5.01 |
| | KN510 200 μM | 57.73 | 8.05 | 26.29 | 7.05 |
| | Gemcitabine 2.5 μM | 1.52 | 0.48 | 35.87 | 3.75 |
| | KN510 200 μM + | 4.14 | 0.40 | 25.06 | 3.93 |
| | Gemcitabine 2.5 μM | | | | |
| Paclitaxel | Control | 100.00 | 0.00 | 100.00 | 2.49 |
| | KN510 200 μM | 57.73 | 8.05 | 19.25 | 2.23 |
| | Paclitaxel 10 nM | 31.96 | 8.18 | 4.35 | 3.36 |
| | KN510 200 μM + | 22.60 | 5.56 | −25.20 | 5.74 |
| | Paclitaxel 10 nM | | | | |
| Cisplatin | Control | 100.00 | 0.00 | 100.00 | 3.32 |
| | KN510 200 μM | 57.73 | 8.05 | 37.94 | 2.30 |
| | Cisplatin 1 μM | 93.66 | 0.00 | 90.80 | 2.86 |
| | KN510 200 μM + | 74.04 | 15.63 | 36.15 | 1.71 |
| | Cisplatin 1 μM | | | | |

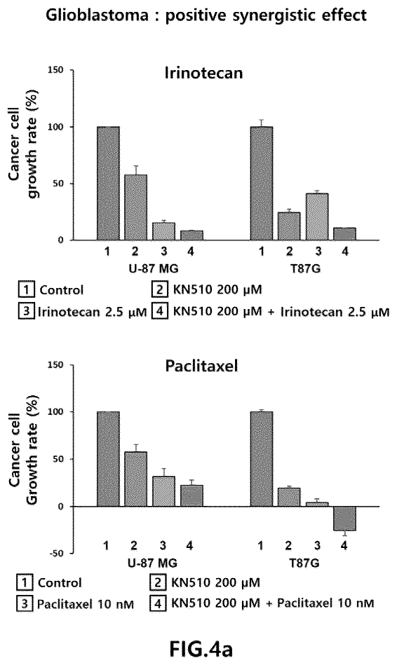

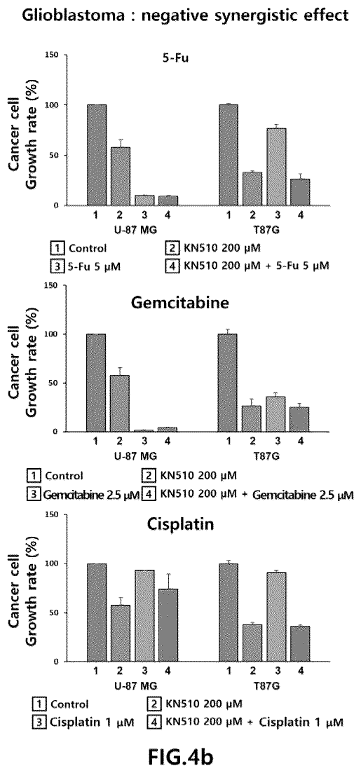

As shown in FIG. 4 and Table 5, when glioblastoma cell lines were treated with irinotecan or paclitaxel in combination with omeprazole (KN510), it was confirmed that glioblastoma cell growth was significantly inhibited compared to the group treated alone.

### Example 5: Confirmation of Anticancer Activity Against Melanoma by Co-Administration

Experiments were performed using the melanoma cell lines UACC62 cells and UACC257 cells in the same manner as in Example 1 above.

TABLE 6
Melanoma cell line growth rate in response to drug treatment

| | | UACC62 | | UACC257 | |
|---|---|---|---|---|---|
| | | average | | average | |
| Drug | | (%) | SD | (%) | SD |
| Irinotecan | Control | 100.00 | 13.71 | 100.00 | 7.53 |
| | KN510 200 μM | 24.68 | 26.65 | 4.33 | 3.12 |
| | Irinotecan 2.5 μM | −35.63 | 3.35 | 21.87 | 4.51 |
| | KN510 200 μM + | −66.29 | 3.21 | −7.67 | 3.38 |
| | Irinotecan 2.5 μM | | | | |
| 5-FU | Control | 100.00 | 11.32 | 100.00 | 7.91 |
| | KN510 200 μM | 24.68 | 26.65 | 10.97 | 0.99 |
| | 5-FU 5 μM | 52.14 | 16.66 | 62.17 | 1.60 |
| | KN510 200 μM | 7.45 | 2.01 | 6.76 | 1.38 |
| | 5-FU 5 μM | | | | |
| Gemcitabine | Control | 100.00 | 6.02 | 100.00 | 5.75 |
| | KN510 200 μM | 42.31 | 12.05 | 3.31 | 1.32 |
| | Gemcitabine 2.5 μM | 53.65 | 12.13 | 30.97 | 2.01 |
| | KN510 200 μM + | 55.00 | 6.80 | 7.58 | 1.60 |
| | Gemcitabine 2.5 μM | | | | |
| Paclitaxel | Control | 100.00 | 14.85 | 100.00 | 0.60 |
| | KN510 200 μM | 38.66 | 5.04 | 3.31 | 0.57 |
| | Paclitaxel 10 nM | 48.36 | 6.74 | 35.43 | 2.84 |
| | KN510 200 μM + | 22.68 | 6.40 | −9.72 | 1.46 |
| | Paclitaxel 10 nM | | | | |
| Cisplatin | Control | 100.00 | 9.07 | 100.00 | 7.92 |
| | KN510 200 μM | 57.65 | 5.41 | 3.10 | 0.26 |
| | Cisplatin 1 μM | 62.76 | 6.38 | 76.16 | 5.77 |
| | KN510 200 μM + | 42.18 | 7.15 | 4.46 | 0.98 |
| | Cisplatin 1 μM | | | | |

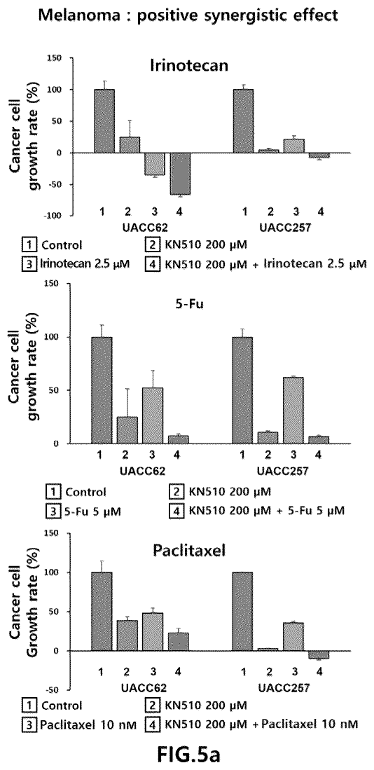

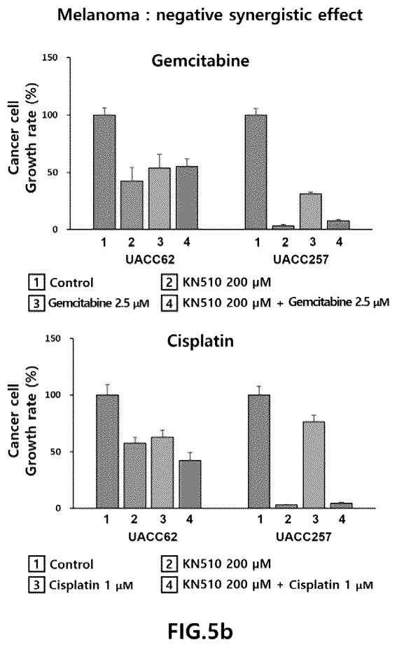

As shown in FIG. 5 and Table 6, when melanoma cell lines were treated with irinotecan, 5-FU, or paclitaxel in combination with omeprazole (KN510), it was confirmed that melanoma cell growth was significantly inhibited compared to the group treated alone.

### Example 6: Confirmation of Anti-Cancer Activity Against Pancreatic Cancer by Co-Administration

Experiments were performed using MIA PaCa-2 cells and PANC-1 cells, a pancreatic ductal adenocarcinoma (PDAC) cell line, in the same manner as in Example 1 above.

TABLE 7
Pancreatic cancer cell line growth
rate in response to drug treatment

| | | MIA PaCa-2 | | PANC-1 | |
|---|---|---|---|---|---|
| | | average | | average | |
| Drug | | (%) | SD | (%) | SD |
| Irinotecan | Control | 100.00 | 7.34 | 100.00 | 22.49 |
| | KN510 200 μM | 32.79 | 3.42 | 41.83 | 7.26 |
| | Irinotecan 2.5 μM | 46.19 | 1.11 | 60.53 | 13.72 |
| | KN510 200 μM + | 21.52 | 1.85 | 34.26 | 2.95 |
| | Irinotecan 2.5 μM | | | | |
| 5-FU | Control | 100.00 | 1.18 | 100.00 | 2.30 |
| | KN510 200 μM | 28.98 | 1.98 | 35.97 | 4.43 |
| | 5-FU 5 μM | 74.96 | 1.74 | 55.46 | 3.77 |
| | KN510 200 μM | 26.00 | 0.39 | 35.83 | 1.47 |
| | 5-FU 5 μM | | | | |
| Gemcitabine | Control | 100.00 | 3.73 | 100.00 | 2.95 |
| | KN510 200 μM | 31.47 | 1.19 | 42.34 | 4.93 |
| | Gemcitabine 2.5 μM | 65.63 | 2.31 | 33.37 | 5.34 |
| | KN510 200 μM + | 33.13 | 4.01 | 30.54 | 3.47 |
| | Gemcitabine 2.5 μM | | | | |
| Paclitaxel | Control | 100.00 | 3.87 | 100.00 | 4.12 |
| | KN510 200 μM | 31.14 | 0.42 | 43.15 | 1.26 |
| | Paclitaxel 10 nM | 26.38 | 1.11 | 16.55 | 2.77 |
| | KN510 200 μM + | 15.70 | 0.99 | 9.19 | 2.42 |
| | Paclitaxel 10 nM | | | | |
| Cisplatin | Control | 100.00 | 13.65 | 100.00 | 4.41 |
| | KN510 200 μM | 29.69 | 2.40 | 47.19 | 4.68 |
| | Cisplatin 1 μM | 90.59 | 0.89 | 94.56 | 3.57 |
| | KN510 200 μM + | 61.28 | 4.04 | 79.97 | 6.06 |
| | Cisplatin 1 μM | | | | |

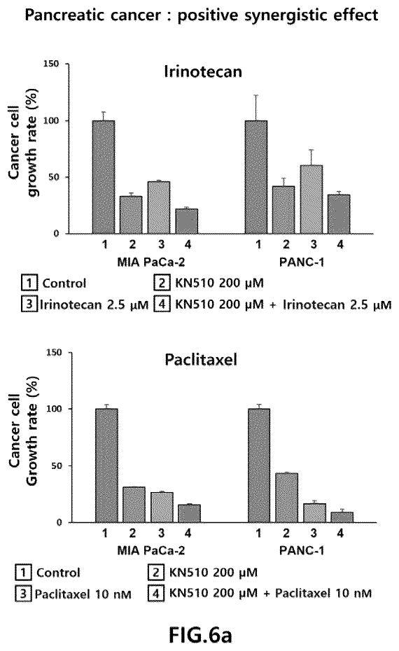

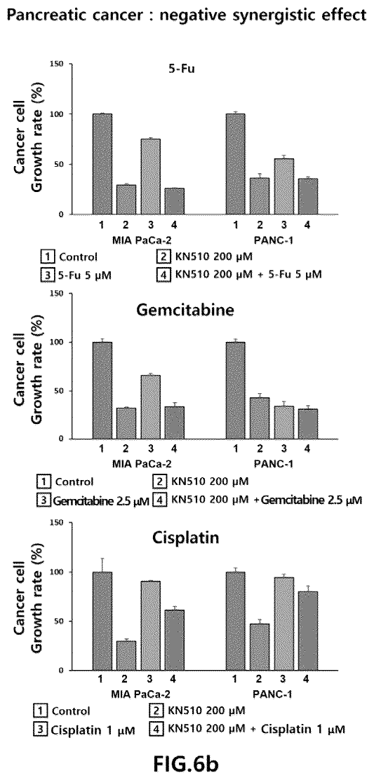

As shown in FIG. 6 and Table 7, when pancreatic cancer cell lines were treated with irinotecan or paclitaxel in combination with omeprazole (KN510), it was confirmed that pancreatic cancer cell growth was significantly inhibited compared to the group treated alone.

### Example 7: Confirmation of Anti-Cancer Activity Against Stomach Cancer by Co-Administration

Experiments were performed using MKN-28 cells and AGS cells, which are stomach cancer cells, in the same way as in Example 1 above.

TABLE 8
Growth rate of stomach cancer cell
lines in response to drug treatment

| | | MKN-28 | | AGS | |
|---|---|---|---|---|---|
| | | average | | average | |
| Drug | | (%) | SD | (%) | SD |
| Irinotecan | Control | 100.00 | 20.30 | 100.00 | 4.46 |
| | KN510 200 μM | 6.43 | 2.05 | 18.91 | 8.02 |
| | Irinotecan 2.5 μM | 76.83 | 9.49 | 55.52 | 13.81 |
| | KN510 200 μM + | 55.18 | 9.80 | 56.96 | 6.93 |
| | Irinotecan 2.5 μM | | | | |
| 5-FU | Control | 100.00 | 8.65 | 100.00 | 3.33 |
| | KN510 200 μM | 7.24 | 1.63 | 26.39 | 1.38 |
| | 5-FU 5 μM | 60.79 | 3.44 | 35.40 | 4.19 |
| | KN510 200 μM | 21.48 | 0.64 | 13.76 | 2.30 |
| | 5-FU 5 μM | | | | |
| Gemcitabine | Control | 100.00 | 4.01 | 100.00 | 6.24 |
| | KN510 200 μM | 11.00 | 4.87 | 33.79 | 7.31 |
| | Gemcitabine 2.5 μM | 42.83 | 2.27 | 10.93 | 2.22 |
| | KN510 200 μM + | 21.30 | 2.13 | 23.57 | 6.38 |
| | Gemcitabine 2.5 μM | | | | |
| Paclitaxel | Control | 100.00 | 2.38 | 100.00 | 2.74 |
| | KN510 200 μM | 9.27 | 1.77 | 31.90 | 4.56 |
| | Paclitaxel 10 nM | 27.71 | 2.65 | 18.60 | 3.22 |
| | KN510 200 μM + | −2.12 | 4.81 | −16.72 | 6.11 |
| | Paclitaxel 10 nM | | | | |
| Cisplatin | Control | 100.00 | 11.90 | 100.00 | 5.83 |
| | KN510 200 μM | 10.82 | 4.62 | 33.59 | 3.07 |
| | Cisplatin 1 μM | 75.14 | 3.02 | 87.44 | 5.24 |
| | KN510 200 μM + | 58.68 | 1.12 | 83.29 | 8.81 |
| | Cisplatin 1 μM | | | | |

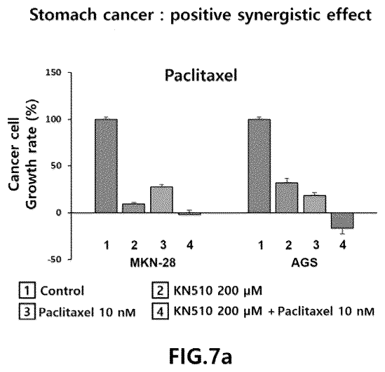

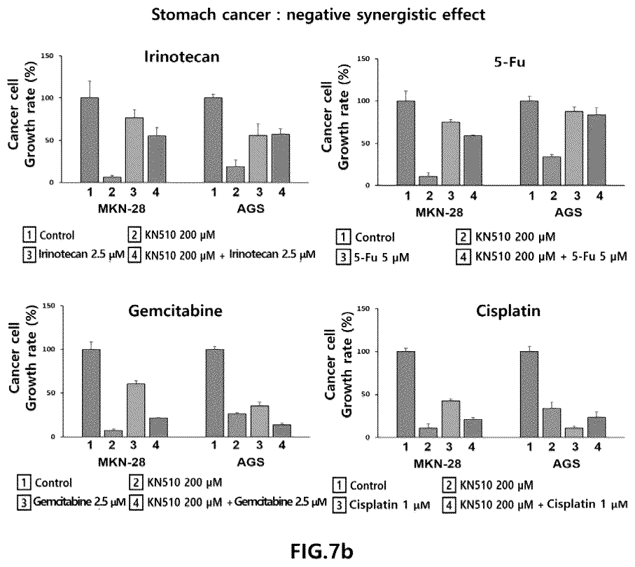

As shown in FIG. 7 and Table 8, when stomach cancer cell lines were treated with paclitaxel in combination with omeprazole (KN510), it was confirmed that stomach cancer cell growth was significantly inhibited compared to the group treated alone.

### Example 8: Confirmation of Anti-Cancer Activity Against Ovarian Cancer by Co-Administration

Experiments were performed using OVCAR-8 cells and SK-OV-3 cells, which are ovarian cancer cells, in the same manner as in Example 1 above.

TABLE 9
Ovarian cancer cell line growth rate in response to drug treatment

| | | OVCAR-8 | | SK-OV-3 | |
|---|---|---|---|---|---|
| | | average | | average | |
| Drug | | (%) | SD | (%) | SD |
| Irinotecan | Control | 100.00 | 4.08 | 100.00 | 9.21 |
| | KN510 200 μM | 38.65 | 1.20 | 58.31 | 14.83 |
| | Irinotecan 2.5 μM | 4.70 | 3.77 | 19.77 | 10.58 |
| | KN510 200 μM + | 1.10 | 0.15 | 17.31 | 9.38 |
| | Irinotecan 2.5 μM | | | | |
| 5-FU | Control | 100.00 | 4.08 | 100.00 | 9.21 |
| | KN510 200 μM | 30.64 | 0.95 | 39.56 | 5.65 |
| | 5-FU 5 μM | 27.12 | 4.09 | 24.31 | 2.12 |
| | KN510 200 μM | 19.09 | 4.52 | 23.67 | 1.57 |
| | 5-FU 5 μM | | | | |
| Gemcitabine | Control | 100.00 | 4.08 | 100.00 | 9.21 |
| | KN510 200 μM | 30.64 | 0.95 | 39.56 | 5.65 |
| | Gemcitabine 2.5 μM | −14.25 | 2.69 | 36.98 | 15.83 |
| | KN510 200 μM + | −12.40 | 4.68 | 30.64 | 15.27 |
| | Gemcitabine 2.5 μM | | | | |
| Paclitaxel | Control | 100.00 | 4.08 | 100.00 | 9.21 |
| | KN510 200 μM | 38.65 | 1.20 | 58.31 | 14.83 |
| | Paclitaxel 10 nM | 12.64 | 0.76 | 32.33 | 0.59 |
| | KN510 200 μM + | 4.52 | 0.92 | 22.03 | 6.08 |
| | Paclitaxel 10 nM | | | | |
| Cisplatin | Control | 100.00 | 4.08 | 100.00 | 9.21 |
| | KN510 200 μM | 30.64 | 0.95 | 39.56 | 5.65 |
| | Cisplatin 1 μM | 51.47 | 7.35 | 43.19 | 8.23 |
| | KN510 200 μM + | 17.37 | 0.58 | 21.61 | 4.66 |
| | Cisplatin 1 μM | | | | |

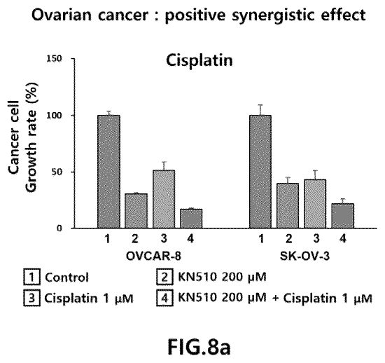

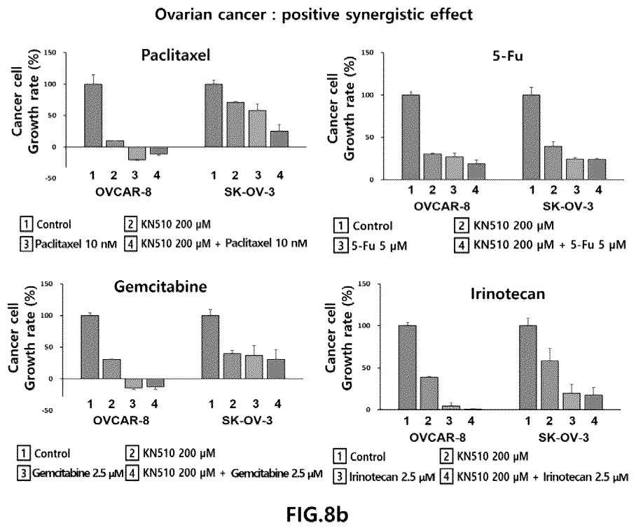

As shown in FIG. 8 and Table 9, when cisplatin was treated in combination with omeprazole (KN510) in ovarian cancer cell lines, it was confirmed that ovarian cancer cell growth was significantly inhibited compared to the group treated alone.

### Example 9: Confirmation of Anti-Cancer Activity Against Lung Cancer by Co-Administration

Experiments were performed in the same manner as in Example 1 above using A549 and H23 lung cancer cells.

TABLE 10
Lung cancer cell line growth rate in response to drug treatment

| | | A549 | | H23 | |
|---|---|---|---|---|---|
| | | average | | average | |
| Drug | | (%) | SD | (%) | SD |
| Irinotecan | Control | 100.00 | 2.54 | 100.00 | 30.63 |
| | KN510 200 μM | 10.47 | 1.34 | 5.67 | 4.03 |
| | Irinotecan 2.5 μM | 16.54 | 2.40 | 39.98 | 2.97 |
| | KN510 200 μM + | 12.55 | 2.04 | −56.49 | 4.06 |
| | Irinotecan 2.5 μM | | | | |
| 5-FU | Control | 100.00 | 2.54 | 100.00 | 30.63 |
| | KN510 200 μM | 10.47 | 1.34 | 6.15 | 0.39 |
| | 5-FU 5 μM | 27.36 | 7.34 | 73.30 | 3.93 |
| | KN510 200 μM | −5.26 | 6.50 | −9.52 | 6.03 |
| | 5-FU 5 μM | | | | |
| Gemcitabine | Control | 100.00 | 2.54 | 100.00 | 30.63 |
| | KN510 200 μM | 10.47 | 1.34 | 1.80 | 4.82 |
| | Gemcitabine 2.5 μM | −16.60 | 2.09 | −23.44 | 6.96 |
| | KN510 200 μM + | 2.22 | 1.80 | −8.03 | 4.93 |
| | Gemcitabine 2.5 μM | | | | |
| Paclitaxel | Control | 100.00 | 2.54 | 100.00 | 30.63 |
| | KN510 200 μM | 10.47 | 1.34 | −10.74 | 1.26 |
| | Paclitaxel 10 nM | 22.67 | 7.09 | 24.94 | 1.49 |
| | KN510 200 μM + | −0.10 | 3.37 | −38.28 | 1.78 |
| | Paclitaxel 10 nM | | | | |
| Cisplatin | Control | 100.00 | 2.54 | 100.00 | 30.63 |
| | KN510 200 μM | 10.47 | 1.34 | 2.18 | 4.84 |
| | Cisplatin 1 μM | 99.46 | 2.12 | 92.69 | 29.47 |
| | KN510 200 μM + | 39.29 | 2.61 | 13.41 | 1.62 |
| | Cisplatin 1 μM | | | | |

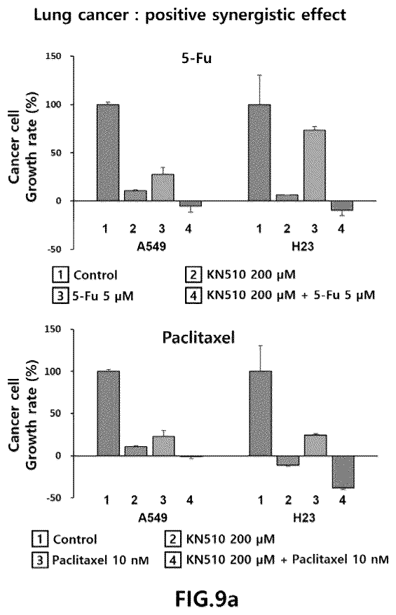

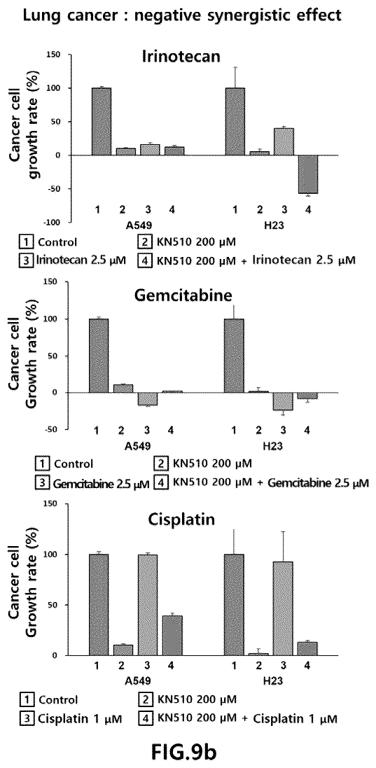

As shown in FIG. 9 and Table 10, when lung cancer cell lines were treated with 5-FU or paclitaxel in combination with omeprazole (KN510), it was confirmed that lung cancer cell growth was significantly inhibited compared to the group treated alone.

### Example 10: Confirmation of Anti-Cancer Activity Against Breast Cancer by Co-Administration

Experiments were performed using breast cancer cells, MDA-MB-231 cells and MCF-7 cells, in the same manner as in Example 1 above.

TABLE 11
Breast cancer cell line growth rate in response to drug treatment

| | | MDA-MB-231 | | MCF-7 | |
|---|---|---|---|---|---|
| | | average | | average | |
| Drug | | (%) | SD | (%) | SD |
| Irinotecan | Control | 100.00 | 3.64 | 100.00 | 3.00 |
| | KN510 200 μM | 38.54 | 2.86 | 31.67 | 2.29 |
| | Irinotecan 2.5 μM | 38.50 | 2.35 | 12.39 | 0.43 |
| | KN510 200 μM + | 2.16 | 1.61 | 1.17 | 0.46 |
| | Irinotecan 2.5 μM | | | | |
| 5-FU | Control | 100.00 | 4.02 | 100.00 | 3.00 |
| | KN510 200 μM | 38.54 | 2.49 | 31.67 | 2.29 |
| | 5-FU 5 μM | 74.97 | 6.07 | 28.09 | 1.10 |
| | KN510 200 μM | 33.01 | 0.34 | 19.68 | 2.71 |
| | 5-FU 5 μM | | | | |
| Gemcitabine | Control | 100.00 | 3.64 | 100.00 | 3.00 |
| | KN510 200 μM | 38.54 | 2.86 | 31.67 | 2.29 |
| | Gemcitabine 2.5 μM | 44.28 | 5.36 | 22.28 | 2.83 |
| | KN510 200 μM + | 28.38 | 1.81 | 14.58 | 0.38 |
| | Gemcitabine 2.5 μM | | | | |
| Paclitaxel | Control | 100.00 | 3.64 | 100.00 | 3.00 |
| | KN510 200 μM | 38.54 | 2.86 | 31.67 | 2.29 |
| | Paclitaxel 10 nM | 55.05 | 1.99 | 35.62 | 2.16 |
| | KN510 200 μM + | 33.01 | 1.28 | 14.58 | 1.42 |
| | Paclitaxel 10 nM | | | | |
| Cisplatin | Control | 100.00 | 3.64 | 100.00 | 3.00 |
| | KN510 200 μM | 38.54 | 2.86 | 31.67 | 2.29 |
| | Cisplatin 1 μM | 91.19 | 1.10 | 94.58 | 0.74 |
| | KN510 200 μM + | 37.93 | 1.81 | 30.16 | 1.36 |
| | Cisplatin 1 μM | | | | |

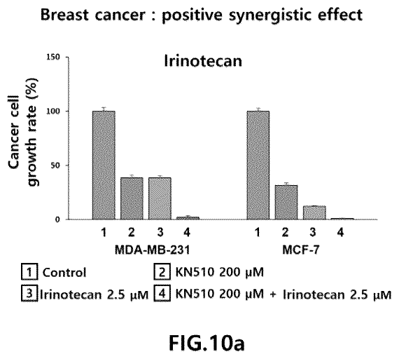

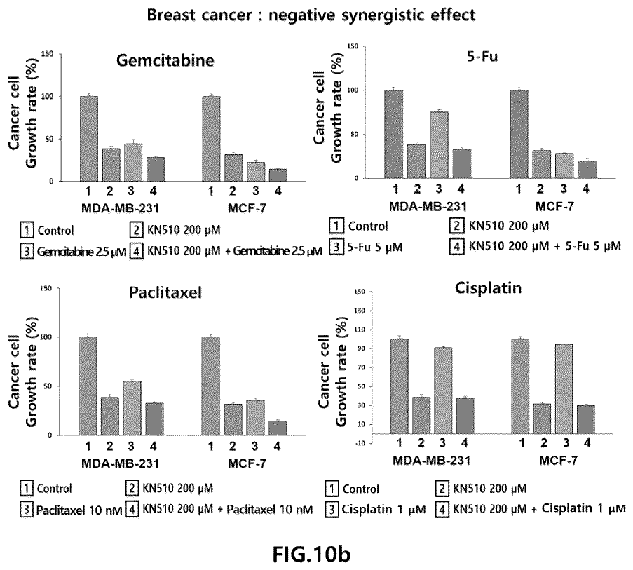

As shown in FIG. 10 and Table 11, when breast cancer cell lines were treated with irinotecan in combination with omeprazole (KN510), it was confirmed that breast cancer cell growth was significantly inhibited compared to the group treated alone.

### Example 11: Confirmation of Anti-Cancer Activity Against Prostate Cancer by Co-Administration

Experiments were performed using prostate cancer cells, PC-3 cells and DU-145 cells, in the same manner as in Example 1 above.

TABLE 12
Prostate cancer cell line growth rate in response to drug treatment

| | | PC-3 | | DU-145 | |
|---|---|---|---|---|---|
| | | average | | average | |
| Drug | | (%) | SD | (%) | SD |
| Irinotecan | Control | 100.00 | 2.99 | 100.00 | 3.16 |
| | KN510 200 μM | 12.70 | 0.86 | 43.66 | 6.14 |
| | Irinotecan 2.5 μM | 33.84 | 0.51 | −17.79 | 1.02 |
| | KN510 200 μM + | 7.18 | 0.31 | −0.15 | 3.99 |
| | Irinotecan 2.5 μM | | | | |
| 5-FU | Control | 100.00 | 2.99 | 100.00 | 3.16 |
| | KN510 200 μM | 12.70 | 0.86 | 43.66 | 6.14 |
| | 5-FU 5 μM | 55.19 | 1.14 | 71.75 | 2.11 |
| | KN510 200 μM | 7.72 | 1.04 | 30.29 | 1.99 |
| | 5-FU 5 μM | | | | |
| Gemcitabine | Control | 100.00 | 2.99 | 100.00 | 3.16 |
| | KN510 200 μM | 12.70 | 0.86 | 43.66 | 6.14 |
| | Gemcitabine 2.5 μM | 32.17 | 0.91 | 0.24 | 0.01 |
| | KN510 200 μM + | 8.95 | 0.46 | 0.36 | 0.01 |
| | Gemcitabine 2.5 μM | | | | |
| Paclitaxel | Control | 100.00 | 2.99 | 100.00 | 3.16 |
| | KN510 200 μM | 12.70 | 0.86 | 43.66 | 6.14 |
| | Paclitaxel 10 nM | 32.49 | 2.10 | 57.24 | 2.82 |
| | KN510 200 μM + | 4.88 | 0.79 | 25.86 | 1.86 |
| | Paclitaxel 10 nM | | | | |
| Cisplatin | Control | 100.00 | 2.99 | 100.00 | 3.16 |
| | KN510 200 μM | 12.70 | 0.86 | 43.66 | 6.14 |
| | Cisplatin 1 μM | 82.60 | 3.31 | 99.32 | 4.79 |
| | KN510 200 μM + | 10.67 | 0.13 | 46.57 | 1.30 |
| | Cisplatin 1 μM | | | | |

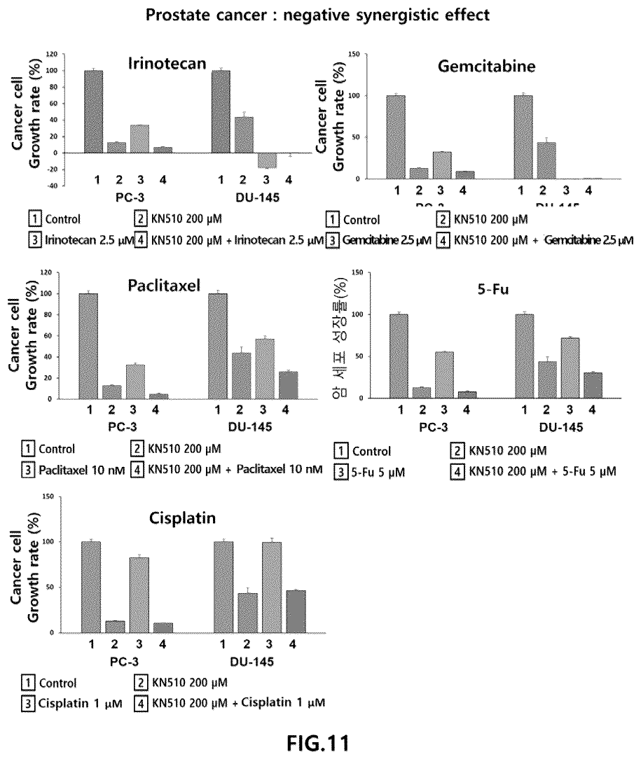

As a result, as shown in FIG. 11 and Table 12, it was confirmed that there was no synergistic effect of combined administration in the case of prostate cancer.

### Example 12: Confirmation of Anti-Leukemic Activity Against Leukemia by Co-Administration

Experiments were performed using leukemia cells, SR cells and K562 cells, in the same manner as in Example 1 above.

TABLE 13
Leukemia cell line growth rate in response to drug treatment

| | | SR | | K562 | |
|---|---|---|---|---|---|
| | | average | | average | |
| Drug | | (%) | SD | (%) | SD |
| Irinotecan | Control | 100.00 | 1.54 | 100.00 | 2.57 |
| | KN510 200 μM | 42.75 | 2.22 | 34.61 | 1.78 |
| | Irinotecan 2.5 μM | 6.26 | 5.50 | 46.54 | 5.27 |
| | KN510 200 μM + | 1.93 | 15.73 | 9.61 | 3.20 |
| | Irinotecan 2.5 μM | | | | |
| 5-FU | Control | 100.00 | 1.54 | 100.00 | 2.57 |
| | KN510 200 μM | 42.75 | 2.22 | 34.61 | 1.78 |
| | 5-FU 5 μM | 96.15 | 1.84 | 99.83 | 4.16 |
| | KN510 200 μM | 22.63 | 4.01 | 26.44 | 2.19 |
| | 5-FU 5 μM | | | | |
| Gemcitabine | Control | 100.00 | 1.54 | 100.00 | 2.57 |
| | KN510 200 μM | 42.75 | 2.22 | 34.61 | 1.78 |
| | Gemcitabine 2.5 μM | 53.15 | 2.31 | 53.51 | 6.69 |
| | KN510 200 μM + | 32.99 | 8.98 | 25.22 | 4.36 |
| | Gemcitabine 2.5 μM | | | | |
| Paclitaxel | Control | 100.00 | 1.54 | 100.00 | 2.57 |
| | KN510 200 μM | 42.75 | 2.22 | 34.61 | 1.78 |
| | Paclitaxel 10 nM | 94.83 | 6.35 | 59.27 | 3.01 |
| | KN510 200 μM + | 0.71 | 7.95 | 12.44 | 1.65 |
| | Paclitaxel 10 nM | | | | |
| Cisplatin | Control | 100.00 | 1.54 | 100.00 | 2.57 |
| | KN510 200 μM | 42.75 | 2.22 | 34.61 | 1.78 |
| | Cisplatin 1 μM | 93.52 | 2.99 | 102.62 | 1.83 |
| | KN510 200 μM + | 30.20 | 1.23 | 31.86 | 0.26 |
| | Cisplatin 1 μM | | | | |

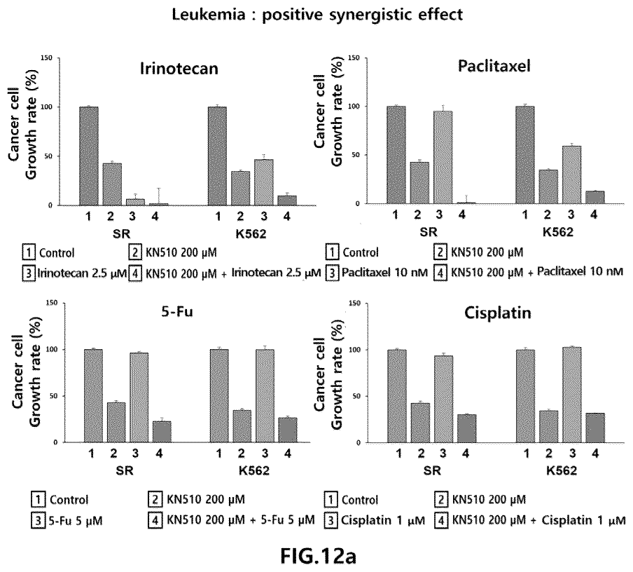

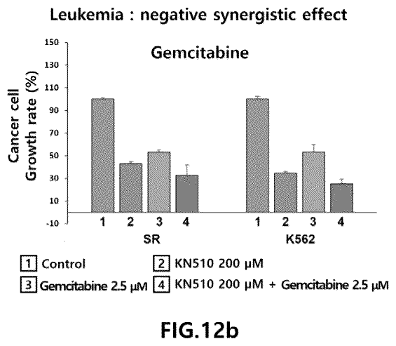

As shown in FIG. 12 and Table 13, when leukemia cell lines were treated with irinotecan, 5-FU, paclitaxel, or cisplatin in combination with omeprazole (KN510), it was confirmed that leukemia cell growth was significantly inhibited compared to the group treated alone.

### Example 13: Confirmation of Anti-Cancer Activity of Omeprazole (KN510) in Combination with Irinotecan in a Xenograft Tumor Model

To determine the anticancer synergistic effects of omeprazole (KN510) and irinotecan co-administration in vivo, a pancreatic cancer xenograft tumor model was constructed and administered with the drugs.

This experiment was reviewed and approved by the Institutional Animal Care and Use Committee (IACUC) of the Korean National Cancer Center Research Institute, which is accredited by the Association for the Accreditation of Laboratory Animal Care and Use (AAALAC International) in accordance with the Guide for the Care and Use of Laboratory Animals (Protocol: NCC-19-494).

Specifically, 6-8 week old Balb/c nude mice (Orient, Korea) were inoculated subcutaneously with MIA PaCa-2 cells (1×10⁷) in 100 μl PBS using 1 ml syringe, and when the tumor size reached 100 mm³, the mice were randomly divided into 4 groups with 4 mice per group, and then treated with drugs as shown in Table 14 below.

We also performed two separate experiments with omeprazole (KN510), one at a concentration of 50 mg/kg and the other at 100 mg/kg.
1) Control (solvent treatment)
2) Omeprazole (KN510) alone (50 mg/kg or 100 mg/kg, P.O. injection),
3) Irinotecan alone (20 mg/kg, I.P injection),
4) Omeprazole (KN510) (50 mg/kg or 100 mg/kg)+irinotecan (20 mg/kg/) as a combination treatment group

TABLE 14
Conditions for medication administration

| | |
|---|---|
| Cell line | MIA PaCa-2 |
| Mice | BALB/c-nu (Orient) |
| N (Head) | 4 |
| Drug delivery | KN510: PO/5 days/week |
| | Irinotecan: IP/1 days/week |
| Treatment on/off | On (5) Off (2) |
| Week | 4 |
| Drug dose | KN510; 50 mg/kg/300 μl or 100 mg/kg/300 μl |
| | Irinotecan: 20 mg/kg/100 μl |
| Drug vehicle | KN510; 5% DMSO + 10% Kolliphor + 85% PBS |
| | (P.O.) Irinotecan; 100% D.W (I.P) |

Primary tumor size was measured weekly using calipers. Tumor volumes were calculated using Equation 1 below, and data are expressed as mean (mean)+standard deviation (SD) values of mouse tumor volume per group.

$$V = ( A \times {B}^{2} ) / 2 ( V = volume ( {mm}^{3} ) , \\ A = long diameter , B = short diameter ) . \qquad [ Equation 1 ]$$

TABLE 15
Tumor volume at 50 mg/kg of omeprazole (KN510) (mm)³

| | 2 week | | 3 week | | 4 week | | 5 week | |
|---|---|---|---|---|---|---|---|---|
| Drug | average | SD | average | SD | average | SD | average | SD |
| Control | 143.54 | 20.72 | 593.43 | 267.26 | 835.78 | 303.81 | 1102.18 | 406.45 |
| KN510 50 mg/kg | 143.01 | 26.83 | 675.69 | 344.08 | 965.63 | 294.97 | 1296.93 | 458.10 |
| Irinotecan 20 | 142.65 | 25.59 | 538.05 | 146.81 | 641.74 | 159.22 | 838.70 | 231.10 |
| mg/kg | | | | | | | | |
| KN510 50 mg/kg + | 138.13 | 43.68 | 382.56 | 278.28 | 351.85 | 221.68 | 314.19 | 329.91 |
| Irinotecan 20 | | | | | | | | |
| mg/kg | | | | | | | | |

TABLE 16
Tumor volume at 100 mg/kg of omeprazole (KN510) (mm)³

| | 2 week | | 3 week | | 4 week | | 5 week | |
|---|---|---|---|---|---|---|---|---|
| Drug | average | SD | average | SD | average | SD | average | SD |
| Control | 98.14 | 9.05 | 412.10 | 74.88 | 907.60 | 174.78 | 1361.54 | 409.43 |
| KN510 100 mg/kg | 93.96 | 36.90 | 229.95 | 59.83 | 469.90 | 219.87 | 843.25 | 371.66 |
| Irinotecan 20 mg/kg | 89.18 | 18.51 | 271.99 | 114.54 | 487.26 | 312.91 | 551.75 | 349.83 |
| KN510 100 mg/kg + | 93.62 | 24.28 | 172.67 | 59.91 | 350.35 | 176.91 | 303.99 | 231.96 |
| Irinotecan 20 mg/kg | | | | | | | | |

As a result, as shown in FIG. 13, Table 15, and Table 16, it was confirmed that when omeprazole (KN510) and irinotecan were administered together, the size of the tumor was significantly reduced compared to the single treatment group.

The composition comprising the carnitine acylcarnitine carrier inhibitor and the anticancer agent of the present invention not only significantly reduced the growth of various cancer cells compared to the use of the carnitine acylcarnitine carrier inhibitor or the anticancer agent alone, but also confirmed the anti-cancer synergistic effect of co-administration of the carnitine acylcarnitine carrier inhibitor and the anticancer agent in a xenograft tumor animal model. Thus, the composition of the present invention is useful as an effective combination anticancer agent.

1. A pharmaceutical composition for the prevention or treatment of cancer, comprising a Carnitine Acylcarnitine Carrier (CAC) inhibitor and an anticancer agent as active ingredients.

2. The pharmaceutical composition for the prevention or treatment of cancer of claim 1, wherein the carnitine acylcarnitine carrier inhibitor is one or more selected from the group consisting of omeprazole (KN510), lansoprazole (KN511), pantoprazole (KN512) and pharmaceutically acceptable salts thereof.

3. The pharmaceutical composition for the prevention or treatment of cancer of claim 1, wherein the anticancer agent is one or more selected from the group consisting of Irinotecan, fluorouracil (5-FU), Paclitaxel, Gemcitabine, Cisplatin and pharmaceutically acceptable salts thereof.

4. The pharmaceutical composition for the prevention or treatment of cancer of claim 1, wherein the composition comprises the carnitine acylcarnitine carrier inhibitor and the anticancer agent in a concentration ratio of 100:0.1 to 1:1.

5. The pharmaceutical composition for the prevention or treatment of cancer of claim 1, wherein the carnitine acylcarnitine carrier inhibitor and the anticancer agent are administered sequentially or simultaneously.

6. The pharmaceutical composition for the prevention or treatment of cancer of claim 1, wherein the cancer is at least one cancer selected from the group consisting of colorectal cancer, lung cancer, stomach cancer, breast cancer, melanoma, leukemia, ovarian cancer, renal cancer, pancreatic cancer, glioblastoma, and liver cancer.

7. The pharmaceutical composition for the prevention or treatment of cancer of claim 1, wherein
when the anticancer agent is irinotecan or a pharmaceutically acceptable salt thereof, the cancer is colon cancer, renal cancer, liver cancer, glioblastoma, pancreatic cancer, breast cancer, or leukemia;
when the anticancer agent is paclitaxel or a pharmaceutically acceptable salt thereof, the cancer is colon cancer, glioblastoma, melanoma, pancreatic cancer, stomach cancer, lung cancer, or leukemia;
when the anticancer agent is 5-FU (fluorouracil) or a pharmaceutically acceptable salt thereof, the cancer is colon cancer, renal cancer, liver cancer, melanoma, lung cancer, or leukemia; and/or
when the anticancer agent is cisplatin or a pharmaceutically acceptable salt thereof, the cancer may be ovarian cancer or leukemia.

8. An anti-cancer adjuvant comprising a Carnitine Acylcarnitine Carrier (CAC) inhibitor and an anti-cancer agent as active ingredients.

9. The anti-cancer adjuvant of claim 8, wherein the carnitine acylcarnitine carrier inhibitor is one or more selected from the group consisting of omeprazole (KN510), lansoprazole (KN511), pantoprazole (KN512) and pharmaceutically acceptable salts thereof.

10. The anti-cancer adjuvant of claim 8, wherein the anti-cancer agent is one or more selected from the group consisting of Irinotecan, fluorouracil (5-FU), Paclitaxel, Gemcitabine, Cisplatin and pharmaceutically acceptable salts thereof.

11. The anti-cancer adjuvant of claim 8, wherein the adjuvant comprises the carnitine acylcarnitine carrier inhibitor and the anticancer agent in a concentration ratio of 100:0.1 to 1:1.

12. The anti-cancer adjuvant of claim 8, wherein the carnitine acylcarnitine carrier inhibitor and the anticancer agent are administered sequentially or simultaneously.

13. The anti-cancer adjuvant of claim 8, wherein the cancer is one or more cancers selected from the group consisting of colon cancer, lung cancer, stomach cancer, breast cancer, melanoma, leukemia, ovarian cancer, renal cancer, pancreatic cancer, glioblastoma, and liver cancer.

14. The anti-cancer adjuvant of claim 8, wherein
when the anticancer agent is irinotecan or a pharmaceutically acceptable salt thereof, the cancer is colon cancer, renal cancer, liver cancer, glioblastoma, pancreatic cancer, breast cancer, or leukemia;
when the anticancer agent is paclitaxel or a pharmaceutically acceptable salt thereof, the cancer is colon cancer, glioblastoma, melanoma, pancreatic cancer, stomach cancer, lung cancer, or leukemia;
when the anticancer agent is 5-FU (fluorouracil) or a pharmaceutically acceptable salt thereof, the cancer is colon cancer, renal cancer, liver cancer, melanoma, lung cancer, or leukemia; and/or
when the anticancer agent is cisplatin or a pharmaceutically acceptable salt thereof, the cancer may be ovarian cancer or leukemia.

15. A method for preventing or treating of cancer, comprising administering a Carnitine Acylcarnitine Carrier (CAC) inhibitor and an anticancer agent as active ingredients to a subject in need thereof.
